SEQUENCE LISTING (7 sequences)

SEQ ID NO: 1 - protein, 9 aa, synthetic construct
GLCTLVAML

SEQ ID NO: 2 - protein, 9 aa, synthetic construct
IVTDFSVIK

SEQ ID NO: 3 - protein, 9 aa, synthetic construct
ATIGTAMYK

SEQ ID NO: 4 - protein, 9 aa, synthetic construct
NLVPMVATV

SEQ ID NO: 5 - protein, 27 aa, synthetic construct
WPLLVFLLPACLYLFASCCAHTFSSMS

SEQ ID NO: 6 - protein, 27 aa, synthetic construct
KSLRVQKIRPSILDCNILRVEYSLLIY

SEQ ID NO: 7 - protein, 27 aa, synthetic construct
LVIPLVELSAKQVTFHIPFEVVEKVYP

(19) United States
(12) Patent Application Publication
(10) Pub. No.: US 2023/0348856 A1
(43) Pub. Date: Nov. 2, 2023

(54) PHARMACEUTICAL COMPOSITION, AND PREPARATION METHOD THEREFOR AND APPLICATION THEREOF

(71) Applicant: BGI SHENZHEN, Shenzhen (CN)

(72) Inventors: Yuping GE, Shenzhen (CN); Yaling HUANG, Shenzhen (CN); Zhengqi ZHAO, Shenzhen (CN); Xuan DONG, Shenzhen (CN); Ying GU, Shenzhen (CN); Yong HOU, Shenzhen (CN); Xiuqing ZHANG, Shenzhen (CN)

(21) Appl. No.: 18/190,199

(22) Filed: Mar. 27, 2023

Related U.S. Application Data
(63) Continuation of application No. PCT/CN2020/118108, filed on Sep. 27, 2020.

(51) Int. Cl. C12N 5/0784

(52) U.S. Cl. CPC C12N 5/0639; C12N 2510/00; C12N 2320/31

(57) ABSTRACT

A method for activating an adaptive immune response by adding allogeneic dendritic cells (DCs) and/or viral antigen peptides to conventional DC vaccines to expand the DC vaccine antigen spectrum with the aid of exogenous DC effect, thereby enhancing the anti-tumor effect of the DC vaccine.

### CROSS-REFERENCE TO RELATED APPLICATION(S)

This application is a continuation of International application No. PCT/CN2020/118108, filed on Sep. 27, 2020, the entire disclosure of which is incorporated by reference herein.

### SEQUENCE LISTING

The instant application contains a Sequence Listing which has been submitted in .xml format and is hereby incorporated by reference in its entirety. Said .xml copy was created Jun. 16, 2023, is named “096151_00019_ST26.xml”, and is 7,168 bytes in size.

### TECHNICAL FIELD

The present disclosure relates to the field of cell engineering, and particularly, to a pharmaceutical composition, a preparation method therefor, and uses thereof. More particularly, the present disclosure relates to a pharmaceutical composition, dendritic cells (DCs), a method for preparing the pharmaceutical composition or DCs, a method for treating tumor patients, and a method for improving an immune killing activity of DCs.

### BACKGROUND

The present disclosure relates to the field of cellular immunotherapy, and particularly, relates to a DC composition or vaccine and a preparation method therefor. The composition or vaccine can be used in tumor immunotherapy and individualized precision immunotherapy.

Tumor immunotherapy is one of the research focuses in the medical field and has become another important cancer treatment method besides surgery, radiotherapy and chemotherapy. More and more studies have confirmed that the regression of cancer or tumor lesions can be effectively promoted and thus tumor recurrence and metastasis can be prevented by activating the body's immune system to generate a durable or specific anti-tumor immune response. The body's immune system plays a vital role in the occurrence and development of malignant tumors. Therefore, if the body's immune system can be effectively controlled, tumors can be better prevented and controlled.

DCs are the most powerful antigen-presenting cells (APCs) in the body, which can take up, process, and present in vivo and in vitro antigens to CD4⁺ and CD8⁺ T lymphocytes, thereby inducing a body-specific immune response. Due to the unique role of DCs in inducing the body's immune response, DC-based immunotherapy has attracted more and more attention in clinical applications in recent years, and has become a research focus in China and abroad.

More and more studies have indicated that the tumor-associated antigens or cancer-testis antigens such as alpha-fetoprotein (AFP), melanoma-associated antigen gene MAGE-A3, and tumor-testis antigen NY-ESO-1 are used as vaccines, or DCs loaded with any one of the above antigen peptides are used as a vaccine, to activate the body's immune system, thereby effectively improving the prognosis of tumor patients. For example, Palmer et al. demonstrated that an autologous DC vaccine loaded with liver cancer whole-cell antigens (i.e., tumor cell lysate) can treat patients with advanced primary liver cancer to a certain extent, and 28% of the patients achieved partial remission or stable disease after the treatment, and 23.5% of the patients had significantly decreased maternal serum alpha-fetoprotein (AFP) level after the treatment. At present, three anti-tumor DC vaccines have been approved and marketed worldwide under the trade names of sipuleucel-T (Dendreon, USA), CreaVaxRCC (CreaGene, Korea), and Hybricell (Genoa Biotechnologia, Brazil), indicating the important research value and promising clinical application of tumor immunotherapy represented by DC vaccines.

The tumor antigen-loaded DC vaccine is one of the safe and effective tumor immunotherapies. However, further analysis revealed that, the DC vaccine, despite of its benefits to some patients, has limited and uncertain overall treatment effect. At present, antigen peptides used for preparing DC vaccines are mainly single antigens (e.g., MAGE, NY-ESO-1, and GPC3), which have a single anti-tumor spectrum and limited antigen immunogenicity. Thus, T lymphocytes, which are activated and generated after vaccination, are merely partially active in killing, which is insufficient to eradicate tumor cells with complex antigenic heterogeneity, and they can hardly inhibit tumor growth. In another aspect, in the process of occurrence and development of tumors, a complex immune escape mechanism is gradually established to resist the body's immune attack, such as defective expression of tumor antigens/antigenic modulation, and downregulation or loss of expression of MHC-I molecules and adhesion/costimulatory molecules on the surface of tumor cells, all of which make DCs unable to effectively present antigens in vivo, and unable to provide sufficient activation signals to activate the body's immune system.

Therefore, it is urgent to further improve the functions of conventional anti-tumor DC vaccines to maximize their effects.

### SUMMARY

An objective of the present disclosure is to solve at least one of the technical problems in the prior art.

In the present disclosure, viral antigen peptides and/or low-dose allogeneic DCs, which serve as a non-specific immunopotentiator or immune adjuvant, are added to a conventional DC vaccine to trigger the body's adaptive immune response with aid of the viral antigens and the exogenous DCs, thereby inducing a more effective T-cell response and enhancing the body's immune response to the DC vaccine. The method is efficient and simple, and greatly improves the anti-tumor effect of the DC vaccine, thereby providing the guarantee for the clinical application of the DC vaccine.

In a first aspect of the present disclosure, the present disclosure provides a pharmaceutical composition. According to embodiments of the present disclosure, the pharmaceutical composition includes autologous DCs and allogeneic DCs. It should be noted that the “autologous DCs” described herein refer to DCs derived from an individual in need of the pharmaceutical composition. For example, if the pharmaceutical composition needs to be administrated to a tumor patient, the autologous DCs in the pharmaceutical composition refer to DCs derived from the tumor patient. Accordingly, the “allogeneic DCs” described herein refer to DCs derived from an individual not in need of the pharmaceutical composition. It should be noted that the “individual not in need of the pharmaceutical composition” and the “individual in need of the pharmaceutical composition” herein are allogeneic. For example, the “allogeneic DCs” are derived from a healthy individual. The body's immune response to the pharmaceutical composition according to the embodiments of the present disclosure is significantly improved.

According to the embodiments of the present disclosure, the above pharmaceutical composition can further include at least one of the following additional technical features.

According to the embodiments of the present disclosure, the autologous DCs are derived from a tumor patient, and the allogeneic DCs are derived from a healthy individual. The pharmaceutical composition according to the embodiments of the present disclosure has a significantly improved immune killing effect on tumor cells.

According to the embodiments of the present disclosure, a ratio of the number of autologous DCs to the number of the allogeneic DCs is (20:1) to (3:1). The Applicant has found that the autologous DCs and the allogeneic DCs in the above ratio range can significantly enhance the body's immune response.

According to the embodiments of the present disclosure, the autologous DCs are loaded with tumor antigen peptides and/or viral antigen peptides. The tumor antigen peptides include patient-individualized neoantigens, tumor-associated antigens (TAAs), and tumor-specific antigens (TSAs). The DCs loaded with the tumor antigen peptides can effectively activate the generation of T lymphocytes, to kill tumor cells. Furthermore, the Applicant has found that the DCs further loaded with the viral antigen peptides can more effectively induce a T-cell response and intensify the body's immune response to the pharmaceutical composition.

According to the embodiments of the present disclosure, the viral antigen peptides include at least one selected from the group consisting of Epstein-Barr virus (EBV) antigen peptides and cytomegalovirus (CMV) antigen peptides.

According to a specific embodiment of the present disclosure, the viral antigen peptide has an amino acid sequence set forth in any one of SEQ ID NO: 1 to SEQ ID NO: 4:

(SEQ ID NO: 1)
GLCTLVAML;
(SEQ ID NO: 2)
IVTDFSVIK;
(SEQ ID NO: 3)
ATIGTAMYK;
and
(SEQ ID NO: 4)
NLVPMVATV.

The viral antigen peptide having an amino acid sequence set forth in SEQ ID NO: 1 is referred to as EBV_A2; the viral antigen peptide having an amino acid sequence set forth in SEQ ID NO: 2 is referred to as EBV A11-1; the viral antigen peptide having an amino acid sequence set forth in SEQ ID NO: 3 is referred to as EBV A11-2; and the viral antigen peptide having an amino acid sequence set forth in SEQ ID NO: 4 is referred to as CMVpp65.

In a second aspect of the present disclosure, the present disclosure provides DCs. According to the embodiments of the present disclosure, the DCs are loaded with tumor antigen peptides and viral antigen peptides. The DCs according to the embodiments of the present disclosure can more effectively induce a T-cell response and intensify the body's immune response to the DCs.

According to the embodiments of the present disclosure, the above DCs can further include at least one of the following additional technical features.

According to the embodiments of the present disclosure, the DCs are derived from a tumor patient.

According to the embodiments of the present disclosure, the viral antigen peptides include at least one selected from the group consisting of EBV antigen peptides and CMV antigen peptides.

In a third aspect of the present disclosure, the present disclosure provides a pharmaceutical composition. According to the embodiments of the present disclosure, the pharmaceutical composition includes the foregoing DCs. The pharmaceutical composition according to the embodiments of the present disclosure can trigger the body's adaptive immune response with aid of the viral antigen peptides to induce a more effective T-cell response and enhance the body's immune response to the DC vaccine.

According to the embodiments of the present disclosure, the above pharmaceutical composition can further include at least one of the following additional technical features.

According to the embodiments of the present disclosure, the pharmaceutical composition further includes autologous DCs derived from a tumor patient and/or allogeneic DCs derived from a healthy individual, and the autologous DCs derived from the tumor patient are loaded with tumor antigen peptides. The pharmaceutical composition according to the embodiments of the present disclosure can induce a more effective T-cell response.

In a fourth aspect, the present disclosure provides use of the foregoing pharmaceutical composition or DCs in preparation of a drug for treating or preventing cancer. It should be noted that the “drug” described herein should be understood in a broad sense, which may be a vaccine for prevention or a drug for treatment. For example, the pharmaceutical composition or DCs according to the embodiments of the present disclosure can be used in the preparation of DC vaccines for preventing cancer, and can also be used in preparation of drugs for treating cancer.

In a fifth aspect of the present disclosure, the present disclosure provides a method for preparing the foregoing pharmaceutical composition. According to the embodiments of the present disclosure, the method includes the following steps: (1) obtaining immature DCs derived from a tumor patient and immature DCs derived from a healthy individual by conducting induced differentiation culturing of CD14+ cells derived from the tumor patient and the3 healthy individual, respectively; (2) obtaining mature DCs derived from the tumor patient by subjecting the immature DCs derived from the tumor patient to tumor antigen peptide loading treatment and mature-induction treatment; (3) obtaining mature DCs derived from the healthy individual by subjecting the immature DCs derived from the healthy individual to mature-induction treatment; and (4) obtaining the pharmaceutical composition comprising the DCs by mixing the mature DCs derived from the tumor patient and the mature DCs derived from the healthy individual. The method for preparing the pharmaceutical composition according to the embodiments of the present disclosure is simple and efficient, and the prepared pharmaceutical composition can trigger a stronger body's immune response.

According to the embodiments of the present disclosure, the above method can further include at least one of the following additional technical features.

According to the embodiments of the present disclosure, a ratio of the number of the mature DCs derived from the tumor patient to the number of the mature DCs derived from the healthy individual ranges from (20:1) to (3:1).

According to the embodiments of the present disclosure, at step 2, the mature DCs derived from the tumor patient are further loaded with viral antigen peptides.

According to the embodiments of the present disclosure, the viral antigen peptides include at least one selected from the group consisting of EBV antigen peptides and CMV antigen peptides.

In a sixth aspect of the present disclosure, the present disclosure provides a method for preparing the foregoing DCs or pharmaceutical composition. According to the embodiments, the method includes the following steps: (1) obtaining immature DCs derived from a tumor patient by conducting induced differentiation culturing of CD14+ cells derived from the tumor patient; (2) obtaining mature DCs derived from the tumor patient by subjecting the immature DCs derived from the tumor patient to tumor antigen polypeptide loading treatment and mature-induction treatment; and (3) obtaining the DCs or the pharmaceutical composition by subjecting the DCs derived from the tumor patient to viral antigen peptide loading treatment. The method for preparing the pharmaceutical composition according to the embodiments of the present disclosure is simple and efficient, and the prepared DCs or pharmaceutical composition can trigger a stronger body's immune response.

According to the embodiments of the present disclosure, the above method can further include at least one of the following additional technical features.

According to the embodiments of the present disclosure, the mature DCs derived from the tumor patient are subjected to the viral antigen peptide loading treatment by incubating the mature DCs together with viral antigen peptides in 5% CO₂ at 37° C. for 4 to 6 hours; the mature DCs are suspended and cultured in a serum-free medium with a concentration of 1×10⁶ cells/mL; and a concentration of the viral antigen peptides in an incubation system is 1 μM. According to the above method provided in the embodiments of the present disclosure, the loading rate of the viral antigen peptides onto the mature DCs can reach 80-95%.

In a seventh aspect of the present disclosure, the present disclosure provides a method for treating tumor patients. According to the embodiments of the present disclosure, the method includes the following steps: obtaining peripheral blood mononuclear cells (PBMCs) from a tumor patient and/or a healthy individual, and sorting CD14+ cells derived from the PBMCs obtained from the tumor patient and/or the healthy individual; preparing, by the method according to any one of claims 11 to 16, a pharmaceutical composition or DCs from the CD14+ cells; and infusing the pharmaceutical composition or the DCs into the tumor patient. The method according to the embodiments of the present disclosure can effectively kill tumor cells for a long term, and inhibit tumor cell growth to achieve a significant treatment effect.

According to the embodiments of the present disclosure, the above method can further include at least one of the following additional technical features.

According to the embodiments of the present disclosure, prior to said infusing the pharmaceutical composition or the DCs into the tumor patient, the method further includes: obtaining activated and amplified T cells by in vitro co-culturing the pharmaceutical composition or the DCs with autologous T cells derived from the tumor patient; and infusing the T cells into the tumor patient.

In an eighth aspect of the present disclosure, the present disclosure provides a method for improving an immune killing activity of autologous DCs. According to the embodiments of the present disclosure, the method includes: mixing the autologous DCs with allogeneic DCs. The method according to the embodiments of the present disclosure can significantly improve the ability of DCs to activate T cells or stimulate T cell proliferation, and improve the ability of DCs to kill tumor cells.

According to the embodiments of the present disclosure, the above method can further include at least one of the following additional technical features.

According to the embodiments of the present disclosure, the autologous DCs are derived from a tumor patient, and the allogeneic DCs are derived from a healthy individual.

According to the embodiments of the present disclosure, a ratio of the number of autologous DCs to the number of the allogeneic DCs is (20:1) to (3:1).

According to the embodiments of the present disclosure, the autologous DCs are loaded with tumor antigen peptides and/or viral antigen peptides.

According to the embodiments of the present disclosure, the viral antigen peptides include at least one selected from the group consisting of EBV antigen peptides and CMV antigen peptides.

In a ninth aspect of the present disclosure, the present disclosure provides a method for improving the tumor immune killing activity of DCs. According to the embodiments of the present disclosure, the method includes: loading DCs with tumor antigen peptides and viral antigen peptides. The method according to the embodiments of the present disclosure can significantly improve the ability of DCs to activate T cells or stimulate T cell proliferation, and improve the ability of DCs to kill tumor cells.

According to the embodiments of the present disclosure, the viral antigen peptides include at least one selected from the group consisting of EBV antigen peptides and CMV antigen peptides.

Some additional aspects and advantages of the present disclosure will be described below, and some additional aspects and advantages of the present disclosure will become obvious from the following description or can be known by implementing the present disclosure.

### BRIEF DESCRIPTION OF DRAWINGS

The above and/or additional aspects and advantages of the present disclosure will become obvious and can be readily understood from the embodiments described below with reference to the drawings.

FIG. 1 is a flowchart of preparation of a novel individualized DC vaccine according to an embodiment of the present disclosure.

FIG. 2 is a result diagram revealing that T cells activated by a novel DC vaccine according to an embodiment of the present disclosure can produce more IFN-γ, where:
T only represents control T cells that are not treated with a DC vaccine;
DC+T represents that T cells are co-cultured with a conventional DC vaccine;
DC+EBV+T represents that T cells are co-cultured with a conventional DC vaccine added with EBV antigen peptides;
DC+alloDC+T represents that T cells are co-cultured with a conventional DC vaccine added with low-dose allogenic DCs; and
OKT3+T represents T cells activated by OKT3, as a positive control.

FIG. 3 is a result diagram revealing that T cells activated by a novel DC vaccine according to an embodiment of the present disclosure has stronger proliferation ability, where:
T only represents control T cells that are not treated with a DC vaccine;
DC+T represents that T cells are co-cultured with a conventional DC vaccine;
DC+EBV+T represents that T cells are co-cultured with a conventional DC vaccine added with EBV antigen peptides; and
DC+alloDC+T represents that T cells are co-cultured with a conventional DC vaccine added with low-dose allogenic DCs.

FIG. 4 is a result diagram revealing that the T cells activated by a novel DC vaccine according to an embodiment of the present disclosure has higher in-vitro tumor killing activity, where:
T+T2 represents that control T cells are co-cultured with T2 tumor cells;
DC+T+T2 represents that T cells activated by a conventional DC vaccine are co-cultured with T2 tumor cells;
DC+EBV+T+T2 represents that T cells activated by a conventional DC vaccine added with EBV antigen peptides are co-cultured with T2 tumor cells; and
DC+alloDC+T+T2 represents that T cells activated by a conventional DC vaccine added with allogenic DCs are co-cultured with T2 tumor cells.

FIG. 5 is a diagram of tumor growth curves of tumor-bearing mice in different groups according to an embodiment of the present disclosure, where:
Pbs represents a blank control group, in which the mice were infused with PBS;
DC-T represents a control DC-T cell group, in which the mice were infused with T cells activated by a conventional DC vaccine containing no polypeptide;
alloDC-T represents a control allogenic DC-T cell group, in which the mice were infused with T cells activated by allogenic DCs;
peptide-DC-T represents a conventional DC vaccine-activated T cell treatment group, in which the mice were infused with T cells activated by a conventional DC vaccine loaded with polypeptides; and
peptide-alloDC-DC-T represents an allogenic DC-added conventional DC vaccine-activated T cell treatment group, in which the mice were infused with T cells activated by a conventional DC vaccine loaded with polypeptides and added with allogenic DCs.

FIG. 6 is comparison a diagram of tumor inhibition rates of mice in respective groups after the infusion according to an embodiment of the present disclosure, where:
Pbs represents a blank control group, in which the mice were infused with PBS;
DC-T represents a control DC-T cell group, in which the mice were infused with T cells activated by a conventional DC vaccine containing no polypeptide;
alloDC-T represents a control allogenic DC-T cell group, in which the mice were infused with T cells activated by allogenic DCs;
peptide-DC-T represents a conventional DC vaccine-activated T cell treatment group, in which the mice were infused with T cells activated by a conventional DC vaccine loaded with polypeptides; and
peptide-alloDC-DC-T represents an allogenic DC-added conventional DC-activated T cell treatment group, in which the mice were infused with T cells activated by a conventional DC vaccine loaded with polypeptides and added with allogenic DCs.

FIG. 7 illustrates flow cytometry patterns of peripheral blood T cells of mice after infusion according to an embodiment of the present disclosure.

### DESCRIPTION OF EMBODIMENTS

The embodiments of the present disclosure will be described below in detail. Examples of the embodiments are shown in the drawings. Throughout the drawings, the same or similar reference signs represent the same or similar elements or elements having the same or similar functions. The embodiments described below with reference to the drawings are exemplary and only used to explain the present disclosure, and they are not intended to limit the present disclosure.

It should be noted that the terms “first” and “second” are only used for the purpose of description, and should not be understood as indicating or implying relative importance or implying the number of indicated technical features. Thus, a feature defined with “first” or “second” may expressly or implicitly include one or more same features. Further, in the description of the present disclosure, unless otherwise specified, “more” means two or more.

With the existing DC vaccine preparation method, the prepared DC vaccine has limited anti-tumor spectrum and antigen immunogenicity, which affect its the effectiveness and the specificity. In view of this fact, the present disclosure provides a method for enhancing the functions of conventional DC vaccines by adding with viral antigen peptides and/or allogenic DCs, thereby triggering a stronger body's immune response. An objective of the present disclosure is to provide a simple and efficient method for preparing a DC vaccine, which can trigger a stronger body immune response.

In order to achieve the above objective, the present disclosure adopts the following technical solutions. The present disclosure provides a method for triggering an adaptive immune response by adding allogenic DCs and/or viral antigen peptides to a conventional DC vaccine, to expand the antigen spectrum and improve the antigen immunogenicity of the conventional DC vaccine with aid of the alloreactivity of the exogenous DCs, thereby enhancing the anti-tumor effect of the DC vaccine. The method includes the following steps.

(1) Acquisition of autologous and allogenic CD14+ cells: CD14+ cells are respectively sorted from PBMCs from a patient and a healthy volunteer by a magnetic bead method; after being sorted, the cells are respectively resuspended in serum-free DC media (CellGenix® GMP DC Medium, 20801-0500) and counted, and the cell density is adjusted to 5×10⁵ cells/mL.

(2) Incubation of immature autologous and allogenic DCs: the CD14+ cells derived from the patient and the volunteer are respectively coated onto plates at a density of 5×10⁵ cells/mL, and GM-CSF (800 U/mL) and IL-4 (1,000 U/mL) are added to the medium. The cells are incubated in 5% CO₂ at 37° C. for 5 days, and during the incubation, half of the medium is replaced once every other day. When replacing the medium, ½ (or ⅓) of the old medium is carefully taken out of the culture dish or culture flask by suction, transferred into a new centrifuge tube (15 mL), and centrifuged at 400 g for 5 min. After the centrifugation, the supernatant is removed, the cell pellet is collected and then added and uniformly mixed with a fresh medium by repeatedly pipetting. The cells are placed on the original culture dish, and cytokines GM-CSF (800 U/mL) and IL-4 (1,000 U/mL) with the same volume are added.

(3) Loading of neoantigen long peptides individualized for the tumor patient onto the DCs, and mature-induction of the DCs: on the 5th day of the incubation of the immature autologous DCs, the medium is replaced, 10 μM long peptides (27-30 aa) are added to the medium to allow the immature DCs to phagocytize the long peptides, and the cells are incubated in 5% CO₂ at 37° C. for 16 hours, the DCs loaded with the long peptides can activate CD4+ and CD8+ T cells at the same time; on the 6th day, maturation factors TNF-α (40 ng/mL), IL-6 (20 ng/mL), IL-1β (20 ng/mL), PGE2 (100 ng/mL), and PolyIC (5 ug/mL) are added to the medium for the immature DCs to induce the maturation of the DCs.

(4) Loading of neoantigen short peptides individualized for the tumor patient onto the DCs: on the 7th day of the incubation, the DCs are maturated, and it can be observed under a microscope that most of the cells are suspended and have obvious small synapses. In this case, suspended DCs are collected and centrifuged at 400 g for 5 min, the supernatant is removed, the cells are resuspended in a medium and counted to adjust the cell density to 1×10⁶ cells/mL; 1 μM antigen short peptides are added (which can be omitted if a DC vaccine treatment regimen uses DCs loaded with long peptides alone); the cells are incubated in 5% CO₂ at 37° C. for 4 hours, and the DCs loaded with the short peptides can activate CD8+ T cells. After the incubation, the DCs loaded with the peptides are washed with a medium and centrifuged at 400 g for 5 min, and the cell pellet is collected and resuspended in a medium for later use. The DCs obtained at this time can be used as a material to prepare a novel DC vaccine, or co-cultured with autologous T lymphocytes in vitro and then used for later function analysis and flow cytometry identification.

(5) Phenotype detection of DCs through flow cytometry: the DCs obtained at the previous step are stained by the following antibodies: CD80 (PE-Cy7), CD83 (APC), CD86 (PE), HLA-DR (PerCP-Cy5.5), and CD14 (APC-Cy7), respectively, and analyzed by using a flow cytometer to detect the expression of each antibody and the maturity of the DCs.

(6) DC harvesting, and preparation of a final novel DC vaccine: 2 days after the maturation of the DCs, the DC suspension is collected and subjected to microorganism, endotoxin, and mycoplasma testing; the qualified autologous DCs, which do not contain bacteria, mycoplasmas, and endotoxins, are mixed with the allogenic DCs according to a ratio of 20:1, 10:1, 5:1, 3:1, or any ratio ranging from (20:1) to (3:1), to obtain a novel individualized DC vaccine containing the allogenic DCs. After being maturated, the autologous DCs are loaded with relevant viral antigen peptides such as EBV antigen peptides and CMV antigen peptides, and mixed with the allogenic DCs to obtain a novel individualized DC vaccine containing the allogenic DCs and the viral antigen peptides. Similarly, a novel individualized DC vaccine only loaded with the viral antigen peptides and contain no allogenic DCs can also be obtained.

(7) In-vitro function test of the novel DC vaccines: each of the prepared novel DC vaccines is co-cultured with T cells, and one week later, the ability of each of the prepared novel DC vaccines to activate T cells and promote T cell proliferation can be tested in vitro. The DC-T cells are co-cultured with corresponding tumor cells, and the tumor killing effect and the IFN-γ production capacity of the DC-T cells can be tested in vitro.

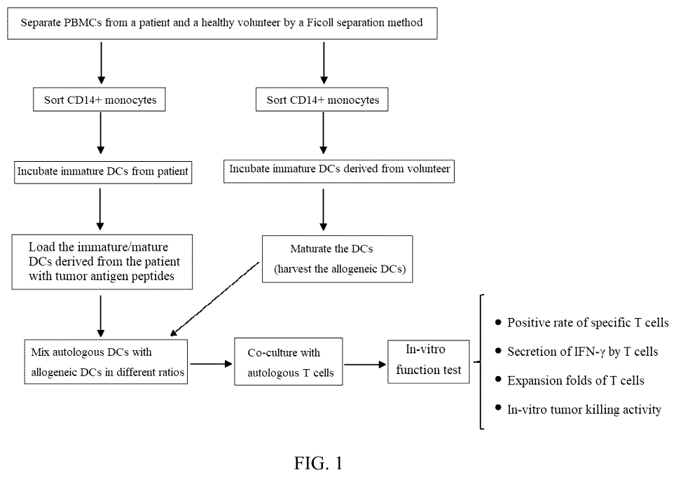

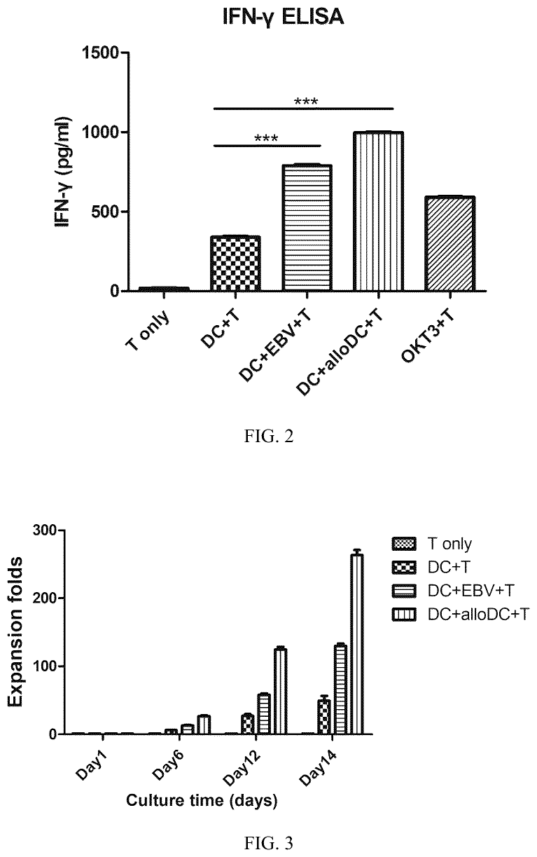

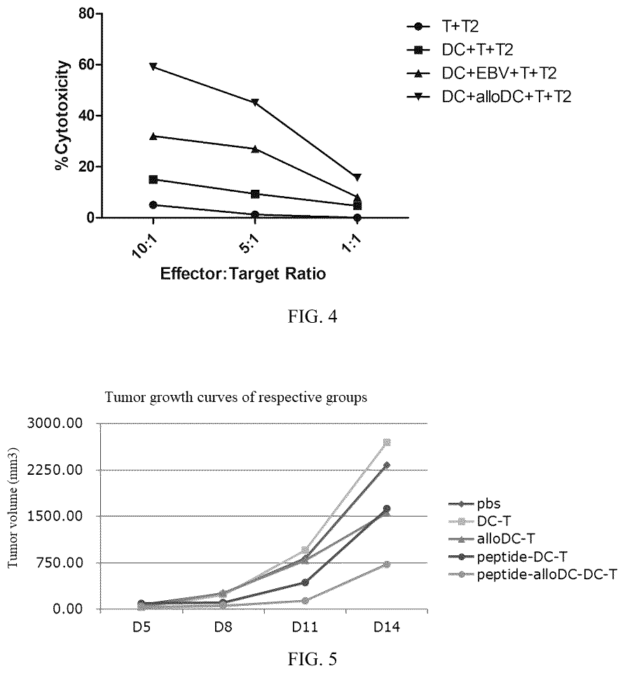

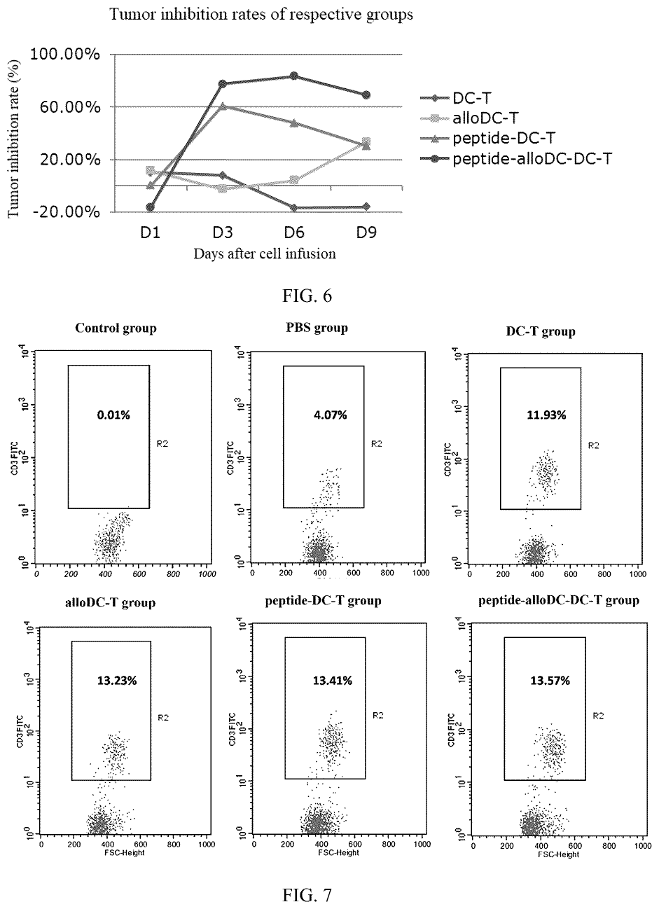

For ease of understanding, the preparation process of the above novel individualized DC vaccine is illustrated in FIG. 1.

Compared with conventional DC vaccines, the ability of the novel DC vaccine prepared by the above method to activate T cells or stimulate T cell proliferation is enhanced by 5 to 50 times, the T cells can be amplified by 200 to 500 times, and the tumor killing activity of effective T cells can be improved by 3-6 times.

Compared with the prior art, the ability of the DCs cultured by the above method to activate T cell or stimulate T cell proliferation as well as the in-vitro tumor killing activity are improved by 3 to 6 times, and the expanding fold of effective T cells is increased by 5 to 50 times. The in vivo assays of mice indicate that the tumor inhibition rate of the DC-T cells cultured by the present method can reach 80% and is greatly higher than that of the conventional DC-T cells, revealing the significant improvement in the effectiveness and the specificity of the DC vaccine.

The technical solutions of the present disclosure will be explained below with reference to examples. Those skilled in the art can understand that the following examples are only used to describe the present disclosure, and are not intended to limit the scope of the present disclosure. In these examples, the unspecified techniques or conditions shall be those described in the literatures (J. Sambrook et al. Molecular Cloning: A Laboratory Manual, Science Press, Third Edition, translated by Huang Peitang et al.) in the related art or the product manual. The reagents or instruments used without specifying manufacturers shall be the commercially available products, for example, purchased from Miltenyi Biotec.

### EXAMPLE 1: CD14+ CELL SORTING

PBMCs from a HLA0201 colorectal tumor patient and a healthy volunteer were obtained by a Ficoll lymphocyte separation method (the subtype of HLA was not specified), CD14+ cells were separated by a magnetic bead method. The specific steps were as follows: 1) the blood sample was transferred into a centrifuge tube (50 mL) and diluted by adding DPBS of the same volume, and the mixture was uniformly mixed by slightly and repeatedly pipetting with a transfer pipette; 2) 18 mL of Ficoll separation solution was taken by suction and transferred to a centrifuged tube (50 mL) for later use; 3) 20 mL of diluted blood sample was slowly placed into the centrifuge tube containing the Ficoll separation solution to allow the diluted blood sample to lie on the surface of the Ficoll separation solution; 4) the mixture was centrifuged at 800 g for 25 min, and after centrifuging, monocytes in the buffer coat were carefully taken by suction and transferred into a sterile centrifuge tube (50 mL); 5) DPBS with a volume 3 times that of the monocyte solution was placed into the centrifuge tube, and uniformly mixed with the monocyte solution by slightly and repeatedly pipetting for several times, and the mixture was centrifuged at 400 g for 10 min; 6) after centrifuging, the supernatant was removed, the cell pellet at the bottom of the tube was slightly flicked, 1 mL of T009 serum-free medium was added to resuspend the cells, and the mixture was centrifuged at 400 g for 10 min; 7) PBMCs were counted, for 1×10⁷ cells, 20 μL of CD14 sorting magnetic beads and 80 μL of MACS buffer (buffer solution) were added and uniformly mixed with the cell mixture, the cell mixture was incubated in a refrigerator in dark at 4° C. for 15 min; 8) after the incubation, for 1×10⁷ cells, 1 to 2 mL of MACS buffer was added to wash the cells, and the mixture was centrifuged at 300 g for 10 min; and 9) the cell pellet was resuspended in 1 mL of MACS buffer, and screened by using a magnetic sorter, a unlabeled discharged cell suspension and labelled cells in the sorting column were respectively collected, and the cells were counted for later use.

### EXAMPLE 2: CULTURING OF DCS

1) Stimulation differentiation of CD14+ cells: the CD14+ cells derived from the colorectal tumor patient and the healthy volunteer, after being sorted, were coated onto plates according to a density of 5×10⁵ cells/mL, respectively, and GM-CSF (800 U/mL) and IL-4 (1,000 U/mL) were added to the medium. The cells were incubated in 5% CO₂ at 37° C. for 5 days, and during the incubation, half of the medium was replaced every other day. When replacing the medium, ½ (or ⅓) of the old medium was carefully taken out of the culture dish or culture flask by suction, transferred into a new centrifuge tube (15 mL), and centrifuged at 400 g for 5 min. After the centrifugation, the supernatant was removed to collect the cell pellet, and the cell pellet was added and uniformly mixed with a fresh medium with the same volume by repeatedly pipetting. The cells were placed into the original culture dish, and cytokines GM-CSF (800 U/mL) and IL-4 (1,000 U/mL) with the same volume were added.

2) Maturation of DCs and loading of polypeptides: on the 5th day of incubation of the immature autologous DCs, the medium was replaced, 10 μM individualized neoantigen long peptides IC-1, IC-2 or IC-3 for the colorectal tumor patient was added to the medium (the amino acid sequences of IC-1, IC-2, and IC-3 are set forth as: IC-1: WPLLVFLLPACLYLFASCCAHTFSSMS (SEQ ID NO:5); IC-2: KSLRVQKIRPSILDCNILRVEYSLLIY (SEQ ID NO:6); and IC-3: LVIPLVELSAKQVTFHIPFEVVEKVYP (SEQ ID NO:7)) to allow the immature DCs to phagocytize the long peptides, and the cells were incubated in 5% CO₂ at 37° C. for 16 hours; and on the 6th day, maturation factors TNF-α (40 ng/mL), IL-6 (20 ng/mL), IL-10 (20 ng/mL), PGE2 (100 ng/mL), and PolyIC (5 μg/mL) were added to the medium for the immature DCs to induce the maturation of the DCs. The DCs derived from the healthy volunteer were not loaded with polypeptides, and on the 6th day, maturation factors TNF-α (40 ng/mL), IL-6 (20 ng/mL), IL-1β (20 ng/mL), PGE2 (100 ng/mL), and PolyIC (5 μg/mL) were added to maturate the DCs for later use.

3) Loading of viral antigen peptides onto the autologous DCs (optionally): on the 7th day of incubation, the DCs were maturated, suspended DCs were collected and centrifuged at 400 g for 5 min; the supernatant was removed, and the cell pellet was resuspended in a serum-free DC medium and counted to adjust the cell density to 1×10⁶ cells/mL; 1 μM viral antigen peptides EBV_A2 or CMVpp65 were added (the amino acid sequences of EBV_A2 and CMVpp65 are ser forth as: EBV_A2: GLCTLVAML (SEQ ID NO:1), and CMVpp65: NLVPMVATV (SEQ ID NO:4)), and the cells were incubated in 5% CO₂ at 37° C. for 4 hours. After incubation, the DCs loaded with the peptides were washed with a medium and centrifuged at 400 g for 5 min, and the cell pellet was collected and resuspended in a medium for later use.

### EXAMPLE 3: PHENOTYPE DETECTION OF THE DCS

The DCs obtained at the previous step were stained with the following antibodies: CD80 (PE-Cy7), CD83 (APC), CD86 (PE), HLA-DR (PerCP-Cy5.5), and CD14 (APC-Cy7), respectively, and then analyzed by using a flow cytometer to detect the expression of each antibody and the maturity of the DCs.

### EXAMPLE 4: PREPARATION OF A FINAL DC VACCINE

2 days after the maturation of the DCs, a DC suspension was collected and subjected to microorganism, endotoxin, and mycoplasma tests; the qualified autologous DCs derived from the colorectal tumor patient, which did not contain bacteria, mycoplasmas, and endotoxins, were mixed with the allogenic DCs derived from the healthy volunteer according to a ratio of 20:1, 10:1, or 3:1, to obtain a novel individualized DC vaccine containing the allogenic DCs for the colorectal tumor patient. The autologous DCs derived from the colorectal tumor patient that were loaded with the relevant viral (e.g., EBV and CMV) antigen peptides of Example 2 were mixed with the allogenic DCs derived from the healthy volunteer to obtain a novel individualized DC vaccine containing the allogenic DCs and the viral antigen peptides. Similarly, a novel individualized DC vaccine only loaded with the viral antigen peptides and contain no allogenic DCs was obtained. The expression of cell markers of the different batches of novel DC vaccines is shown in Table 1.

TABLE 1
Expression of Markers of different batches of novel DC vaccines

| Batches | HLA-DR (%) | CD86 (%) | CD80 (%) | CD83 (%) |
|---|---|---|---|---|
| No. 1 | 99.7% | 98.1% | 90.3% | 67.1% |
| No. 2 | 99.8% | 99.2% | 97.1% | 73.8% |
| No. 3 | 99.9% | 99.8% | 98.4% | 75.3% |
| No. 4 | 99.9% | 99.4% | 98.0% | 74.9% |

### EXAMPLE: 5 IN-VITRO FUNCTION TEST

Each of the above prepared novel DC vaccines was co-cultured with autologous T lymphocytes in vitro, and the abilities of each vaccine to activate T cells, promote T cell proliferation, kill tumors, and produce cytokines were tested. Results are shown in FIGS. 2 to FIG. 4. The results indicate that the novel DC vaccines can stimulate T cells to produce more IFN-γ, and T cells stimulated by the novel DC vaccines have stronger proliferation ability and higher in-vitro tumor killing activity.

### EXAMPLE 6: IN-VIVO MOUSE EXPERIMENT

1) Construction of Mouse Models with an Individualized Mutation of a Colorectal Tumor Patient

First, a K562 cell line stably transfected with a specific antigen peptide mine-gene from a colorectal tumor patient was constructed by Wuhan Viraltherapy Technologies Co., Ltd. Under the entrustment of the inventors. Then, the cell line was amplified in a RPMI1640 medium containing 10% FBS in vitro until an appropriate number of cells were obtained, the cells were subcutaneously infused into 6-week-old NSG mice with immunodeficiency according to an inoculum size of 1×10⁷ mine-gene-K562 cells/mouse. After the infusion, the tumor tissue growth was observed duly, about 5 days later, the mouse models with the tumor tissue of 50 to 100 mm³ were selected for later use.

### 2) Grouping and Cell Infusion

The above successfully constructed NSG mouse models were random divided into the following 5 groups, each group included 5 mice, and different cells were infused into the mice according to the experimental scheme:
a. Blank control group: each mouse was injected with 100 μL of PBS via the tail vein;
b. Control DC-T cell group: each mouse was injected with T cells derived from the colorectal tumor patient activated by autologous DCs, i.e., DC-T cells, via the tail vein according to an inoculum size of 2×10⁷ cells/mouse, and the DC-T cells were suspended in 100 μL of PBS;
c. Control allogeneic DC-T cell group: each mouse was injected with T cells derived from the colorectal tumor patient activated by allogeneic DCs, i.e., alloDC-T cells, via the tail vein according to an inoculum size of 2×10⁷ cells/mouse, and the alloDC-T cells were suspended in 100 μL of PBS;
d. Peptide-DC-T cell group: each mouse was injected with T cells derived from the colorectal tumor patient activated by autologous DCs loaded with individualized antigen peptides, i.e., peptide-DC-T cells, via the tail vein according to an inoculum size of 2×10⁷ cells/mouse, and the peptide-DC-T cells were suspended in 100 μL of PBS; and
e. Peptide-alloDC-DC-T cell group: each mouse was injected with T cells derived from the colorectal tumor patient activated by an DC vaccine containing allogeneic DCs and loaded with individualized antigen peptides, i.e., peptide-alloDC-DC-T cells, via the tail vein according to an inoculum size of 2×10⁷ cells/mouse, and the peptide-alloDC-DC-T cells were suspended in 100 μL of PBS.
   3) Testing after Infusion and Result Analysis

Measurement of the diameter of the tumor and the weight of each mouse: after infusion, each mouse was weighed and the diameter of the tumor was measured every 2 days, and after the average volume of the tumor tissues of the mice in the control group was 2,500 mm³, the observation was finished. At the last observation, each mouse in each group was photographed, and the tumors were taken out and weighed. Results are shown in FIG. 5 and FIG. 6. The results indicate that, the T cells activated by the novel DC vaccine has a better tumor inhibition effect; the tumor tissue growth in the mouse infused with the T cells significantly slows down; and a tumor inhibition rate in the mouse infused with the T cells activated by the novel DC vaccine reaches 80%.

Analysis of T cells in the peripheral blood: one week after infusion and at the end of observation, 2 mice were random selected from each group, 20 μL of blood was collected from each mouse via the tail vein, the proportion of human CD3+ T cells in the peripheral blood of each mouse was analyzed by using the flow cytometer. Results are shown in FIG. 2 and FIG. 7. The results indicate that one week or 9 days after infusion, T cells can still be detected from the peripheral blood of each mouse in the treatment groups.

TABLE 2
Proportion of human CD3+ T cells in the peripheral blood
of each mouse in each group determined 9 d after infusion

| | PBS | | | peptide- | peptide- |
|---|---|---|---|---|---|
| Groups | group | DC-T | alloDC-T | DC-T | alloDC-DC-T |
| Proportion | 4.07 | 11.93 | 13.23 | 13.41 | 13.57 |
| (%) of T cells | | | | | |

Pathological observation: the subcutaneous tumor tissue of each mouse in the control group and the treatment groups was stained with hematoxylin and eosin (H&E) and then subjected to TUNEL assay to detect the tumor tissue apoptosis in each group.

Based on the above testing results, the anti-tumor effect was comprehensively evaluated in terms of the tumor growth curve, tumor inhibition rate, distribution of human T cells in the peripheral blood, and tumor tissue apoptosis in each group, etc. According to the present disclosure, the ability of the DC vaccine to activate T cells and promote proliferation of initial T cells can be effectively enhanced by adding low-dose allogeneic DCs or viral antigen peptides to a conventional DC vaccine, thereby enhancing the anti-tumor effect of the DC vaccine and DC-CTL.

Although specific embodiments of the present disclosure have been described in detail, those skilled in the art can understand that various modifications and substitutions of those details can be made based on all the discloses teachings, and these changes shall fall within the scope of protection of the present disclosure. The full scope of the present disclosure is given by the appended claims and any equivalents thereof

In the description, the content described with the terms “one embodiment”, “some embodiments”, “exemplary embodiment”, “example”, “specific example”, “some examples” or the like means that a specific feature, structure, material, or characteristic described in the embodiment or example is included in at least one embodiment or example of the present disclosure. In the description, exemplary representations of the above terms do not necessarily refer to the same embodiment or example. Furthermore, the described specific feature, structure, material or characteristic can be combined in a suitable manner in any one or more of the embodiments or examples.

1. Dendritic cells (DCs), wherein the DCs are loaded with tumor antigen peptides and viral antigen peptides.

2. The DCs according to claim 1, wherein the DCs are derived from a tumor patient.

3. The DCs according to claim 1, wherein the viral antigen peptides comprise at least one selected from the group consisting of EBV antigen peptides and CMV antigen peptides.

4. A pharmaceutical composition, comprising the DCs according to claim 1.

5. The pharmaceutical composition according to claim 4, further comprising:
autologous DCs derived from a tumor patient; and/or
allogeneic DCs derived from a healthy individual.

6. The pharmaceutical composition according to claim 5, wherein a ratio of the number of autologous DCs to the number of the allogeneic DCs ranges from (20:1) to (3:1).

7. A method for preparing the DCs according to claim 1, comprising the following steps:
1) obtaining immature DCs derived from a tumor patient by conducting induced differentiation culturing of CD14+ cells derived from the tumor patient;
2) obtaining mature DCs derived from the tumor patient by subjecting the immature DCs derived from the tumor patient to tumor antigen polypeptide loading treatment and mature-induction treatment; and
3) obtaining the DCs or the pharmaceutical composition by subjecting the DCs derived from the tumor patient to viral antigen peptide loading treatment.

8. The method according to claim 7, wherein the mature DCs derived from the tumor patient are subjected to the viral antigen peptide loading treatment by:
incubating the mature DCs together with viral antigen peptides in 5% CO₂ at 37° C. for 4 to 6 hours, wherein:
the mature DCs are suspended and cultured in a serum-free medium with a concentration of 1×10⁶ cells/mL; and
a concentration of the viral antigen peptides in an incubation system is 1 μM.

9. A method for preparing the pharmaceutical composition according to claim 4, comprising the following steps:
1) obtaining immature DCs derived from a tumor patient by conducting induced differentiation culturing of CD14+ cells derived from the tumor patient;
2) obtaining mature DCs derived from the tumor patient by subjecting the immature DCs derived from the tumor patient to tumor antigen polypeptide loading treatment and mature-induction treatment; and
3) obtaining the DCs or the pharmaceutical composition by subjecting the DCs derived from the tumor patient to viral antigen peptide loading treatment.

10. The method according to claim 9, wherein the mature DCs derived from the tumor patient are subjected to the viral antigen peptide loading treatment by:
incubating the mature DCs together with viral antigen peptides in 5% CO₂ at 37° C. for 4 to 6 hours, wherein:
the mature DCs are suspended and cultured in a serum-free medium with a concentration of 1×10⁶ cells/mL; and
a concentration of the viral antigen peptides in an incubation system is 1

11. A method for treating a tumor patient, comprising:
obtaining peripheral blood mononuclear cells (PBMCs) from a tumor patient and/or a healthy individual, and sorting CD14+ cells derived from the PBMCs obtained from the tumor patient and/or the healthy individual;
preparing, by the method according to claim 7, a pharmaceutical composition or DCs from the CD14+ cells; and
infusing the pharmaceutical composition or the DCs into the tumor patient.

12. The method according to claim 11, further comprising, prior to said infusing the pharmaceutical composition or the DCs into the tumor patient:
obtaining activated and amplified T cells by in vitro co-culturing the pharmaceutical composition or the DCs with autologous T cells derived from the tumor patient; and
infusing the T cells into the tumor patient.

13. A method for improving an immune killing activity of autologous DCs, comprising:
mixing the autologous DCs with allogeneic DCs.

14. The method according to claim 13, wherein:
the autologous DCs are derived from a tumor patient; and
the allogeneic DCs are derived from a healthy individual.

15. The method according to claim 13, wherein a ratio of the number of autologous DCs to the number of the allogeneic DCs ranges from (20:1) to (3:1).

16. The method according to claim 13, wherein the autologous DCs are loaded with tumor antigen peptides and/or viral antigen peptides.

17. The method according to claim 16, wherein the viral antigen peptides comprise at least one selected from the group consisting of EBV antigen peptides and CMV antigen peptides.
